(19) United States
(12) Patent Application Publication
(10) Pub. No.: US 2005/0271718 A1
(43) Pub. Date: Dec. 8, 2005

(54) Sustained release propafenone hydrochloride capsules

(76) Inventor: Bernard Charles Sherman, Toronto (CA)

Correspondence Address: NIXON & VANDERHYE, PC, 901 NORTH GLEBE ROAD, 11TH FLOOR, ARLINGTON, VA, 22203, US

(21) Appl. No.: 11/146,006

(22) Filed: Jun. 7, 2005

(30) Foreign Application Priority Data
Jun. 7, 2004 (CA) 2,469,339

(51) Int. Cl. A61K 9/54

(52) U.S. Cl. 424/458

(57) ABSTRACT

A sustained release capsule for oral administration containing tablets, wherein each tablet comprises 25 mg propafenone hydrochloride.

### BACKGROUND

Propafenone hydrochloride is an antiarrythmic agent sold in the United States and elsewhere under the tradename Rythmol™, in the form of immediate-release tablets in strengths of 150 mg, 225 mg and 300 mg. The usual dosing schedule is 3 times daily.

In early 2004, propafenone hydrochloride also became available in the United States and elsewhere under the tradename Rythmol SR™ in the form of sustained release capsules in strengths of 225 mg, 325 mg and 425 mg. Because the release from Rythmol SR™ is gradual over many hours after ingestion, the dosing schedule for Rythmol SR™ is only twice daily, which is more convenient for the patient.

Rythmol SR™ capsules are made in accordance with the disclosure of U.S. Pat. Nos. 5,681,588. As explained in the disclosure of that patent, the manufacture of sustained release dosage forms usually requires relatively large quantities of excipients (inactive ingredients). This makes it difficult to produce tablets or capsules that contain relatively large amounts of active ingredients but are still small in size. The disclosure further explains that it was surprisingly found that satisfactory sustained release is achieved from microtablets comprising propafenone hydrochloride with little or no excipients added. These microtablets have a height and diameter, which are each 1-3 mm, and the active ingredient is from 81 to 99.9% of the weight of the microtablet. Gelatin capsules are filled with these microtablets.

™—Trademark

The microtablets actually contained in Rythmol SR™ capsules have a diameter of 2 mm, a propafenone hydrochloride content of 6.25 mg, and a total weight of 6.5 mg per microtablet. Because the propafenone hydrochloride content is 6.25 mg per microtablet, it follows that the number of microtablets required per capsule is 36 for 225 mg capsules, 52 for 325 mg capsules, and 68 for 425 mg capsules.

Although Rythmol SR™ capsules provide the desired sustained release, it is relatively expensive to produce large quantities of microtablets on conventional rotary tablet presses. Rotary tablet presses produce a number of tablets per minute that is equal to the number of rotations of the press per minute multiplied by the number of tooling stations. Hence, for production of microtablets needed to fill a given number of capsules, the production time on a tablet press is directly proportional to the number of microtablets needed. Furthermore, the tooling needed to produce microtablets of 2 mm diameter is relatively fragile and easily broken. Tabletting rates can be increased by using multi-tip tooling, but such tooling is relatively expensive and also relatively fragile.

It is thus the objective of the present invention to enable the manufacture of capsules equivalent to Rythmol SR™, but which require fewer tablets per capsule.

### DESCRIPTION OF THE INVENTION

For a slowly dissolving tablet of a given formulation, dissolution rate decreases with increased size. This is because mass of the tablet is proportional to the cube of linear dimension, whereas surface areas are proportional to the square of linear dimension. Hence, surface area to mass ratio is inversely proportional to linear dimension. It thus would be expected that tablets comprised entirely or almost entirely of propafenone hydrochloride and of size larger than 1-3 mm, which is suggested as necessary in U.S. Pat. No. 5,681,588, would exhibit dissolution rates substantially lower than that of the microtablets in Rythmol SR™.

However, it has been surprisingly found that tablets which are substantially larger in size and weight and are comprised entirely or almost entirely of propafenone hydrochloride can be produced having dissolution which is not significantly slower than that of the microtablets in Rythmol SR™ capsules.

Because Rythmol SR™ capsules are available in strengths of 225 mg, 325 mg and 425 mg, all of which are multiples of 25 mg, it is particularly advantageous to use tablets comprising 25 mg of propafenone hydrochloride per tablet. Capsules equivalent to Rythmol SR™ in 225 mg, 325 mg and 435 mg strengths can then be filled with 9, 13, and 17 tablets per capsule, respectively.

Accordingly, compositions of the present invention are sustained release capsules for oral administration containing tablets, wherein each tablet comprises 25 mg of propafenone hydrochloride.

It will be understood that when a weight is specified herein, such as the weight of propafenone hydrochloride per tablet, the weight of an excipient (inactive ingredient), or a total tablet weight, the specified weight is intended to be the nominal or target weight. Actual individual weights will vary somewhat from the nominal weights as a result of production variability.

As aforesaid, given that each tablet comprises 25 mg of propafenone hydrochloride, capsules of strength 225 mg, 325 mg and 425 mg will contain 9, 13 and 17 tablets, respectively.

To enable the capsules to be of acceptably small size, it is desirable that the tablets be as small as possible, and thus that they contain relatively small amounts of excipients (inactive ingredients), if any.

Each tablet comprising 25 mg propafenone hydrochloride will thus preferably have a weight of from 25 mg to not more than 40 mg. The tablet weight will more preferably be from 25 mg to 35 mg, still more preferably from 25 mg to 30 mg, and even more preferably from 25 mg to 28 mg. It will be understood that, if the tablet weight is 25 mg, such tablet is comprised of 25 mg of propafenone hydrochloride with no excipients.

Tablets that are made of only propafenone hydrochloride are relatively soft and friable. It is thus preferable to include in the tablet a small amount of a binder to increase tablet hardness and reduce friability. The binder will preferably be a water-soluble polymer, which may also serve to slightly increase the dissolution rate. Preferred polymers are povidone and copovidone.

It is also preferred, but not necessary, that the tablets comprise a small amount of a lubricant such as, for example, magnesium stearate, to avoid sticking to the tooling in the tabletting process.

The tablets may also contain excipients for other purposes, such as, for example, an excipient to either increase or decrease the dissolution rate, in order to achieve any desired rate.

The invention is illustrated by the following example:

### Example 1

Ingredients were mixed in the following proportions:

| | |
|---|---|
| Propafenone hydrochloride | 25.00 |
| Copovidone | 2.98 |
| Magnesium stearate | 0.02 |
| | 28.00 |

The powder mixture was compacted, and then milled into small granules. The granules were then compressed into tablets of 28 mg weight on a rotary tablet press, using tooling of 9/64″ (3.5 mm) diameter.

Size 00 capsules were then filled with 17 tablets per capsule.

The dissolution rate of these capsules was then compared to that of Rythmol SR™ capsules in USP Apparatus 2 at 50 rpm. It was found that, in phosphate buffer of both pH4.5 and pH6.8, the dissolution rate of the capsules of this example was similar to the dissolution rate of Rythmol SR™ capsules.

1. A sustained release capsule for oral administration containing tablets, wherein each tablet comprises 25 mg propafenone hydrochloride.

2. A capsule of claim 1 wherein the tablet weight is from 25 to 40 mg.

3. A capsule of claim 1 wherein the tablet weight is from 25 to 35 mg.

4. A capsule of claim 1 wherein the tablet weight is from 25 to 30 mg.

5. A capsule of claim 1 wherein the tablet weight is from 25 to 28 mg.

6. A capsule of claim 1 containing 9 tablets.

7. A capsule of claim 1 containing 13 tablets.

8. A capsule of claim 1 containing 17 tablets.

9. A capsule of claim 1 wherein the tablets further comprises a water-soluble binder.

10. A capsule of claim 9 wherein the water-soluble binder is a polymer.

11. A capsule of claim 10 wherein the polymer is povidone.

12. A capsule of claim 10 wherein the polymer is copovidone.
